(19) United States
(12) Patent Application Publication
(10) Pub. No.: US 2003/0187510 A1
(43) Pub. Date: Oct. 2, 2003

(54) Mobile bearing prostheses

(76) Inventor: Edward R. Hyde, Woodside, CA (US)

Correspondence Address: Pennie & Edmonds, LLP, 3300 Hillview Avenue, Palo Alto, CA, 94304, US

(21) Appl. No.: 10/420,648

(22) Filed: Apr. 21, 2003

Related U.S. Application Data
(63) Continuation-in-part of application No. 09/849,379, filed on May 4, 2001, now Pat. No. 6,599,321.

(51) Int. Cl. A61F 2/30

(52) U.S. Cl. 623/18.12

(57) ABSTRACT

A prosthesis includes at least a first component and a second component. The components being disposed with a variety of magnetic material. The magnetic material being either hard magnetic material or soft magnetic material. The magnetic material is arranged and configured to magnetically interact and controllably constrain movement between the components. The magnetic material is also organized to generate a gradient against movement of the components. The magnetic material also forms opposing grid patterns that interlock magnetically.

### RELATED APPLICATION

The present application is a continuation-in-part of U.S. patent application, Ser. No. 09/849,379 filed May 4, 2001, which is incorporated by reference herein in its entirety.

### FIELD OF THE INVENTION

Generally the present invention relates to orthopaedic prostheses. More particularly, the present invention relates to orthopaedic prostheses encompassing a self-constraining mobile bearing for maintaining a desired relationship among joint components, thereby reducing friction, wear, deformation, mechanical loosening, and the like.

### BACKGROUND OF THE INVENTION

Orthopedics is a medical sub-specialty that treats anatomical disorders related to bones, muscles, ligaments, tendons, joints, and the like. A current emphasis in orthopedics is the treatment of the bones and joints as there is a great need in modern society for bone and joint repair and replacement. The treatment of bone and joint disorders can be generally subclassified into categories including the treatment of bone fractures, joint instability, early stage arthritis, and end stage arthritis. Originally, the treatment of orthopedic conditions had mainly relied on casting and bracing. However, with the advent of new implantable materials and development of better joint replacement prostheses, the focus of orthopedics has shifted to become increasingly more of a surgical sub-specialty. With improved materials, better engineering, and a better understanding of the human body, the practice of orthopedic medicine and biomechanical experimentation have made remarkable progress. The treatment of bone fractures and joint disorders has continually been refined to the present state-of-the-art. The last 40 years have shown a myriad of innovations that have concentrated specifically on developing static mechanical design characteristics and new implantable materials used for fracture treatment and in total joint arthroplasties. These static mechanical design characteristics have been directed to solutions for problems concerning wear, stability, and methods of fixation for the total joint arthroplasties. They have also been utilized to improve the current state of the art concerning fracture treatment.

It is now well understood that the design of artificial joints must provide for accommodation of mechanical stresses generated through use of the joint. For example, compression, tension, translation and rotational shear forces are generated at the knee in addition to the articulation rotation when a person moves from sitting to standing. In a normal knee, the articulation surfaces are effectively frictionless, and muscle, ligaments and the meniscus resist and absorb such forces. In artificial joints, materials such as ultra-high molecular weight polyethylene have been employed at bearing surfaces to provide a degree of energy absorption and wear resistance. However, it is not yet possible to provide effectively frictionless articulation surfaces, leading to excessive wear, creation of wear debris and premature loosening of the components. Also, energy absorption between components transfers excessive impact force to the bone-prosthesis interface, contributing to resorption and loosening. For these reasons, attempts have been made to uncouple undesirable mechanical stresses from the articulation surface. In artificial knees, mobile bearings have been introduced for this purpose. A mobile bearing inserted between two components allows motion on its upper and lower surfaces so energy is dissipated rather than confined to the front side where it causes damage. Still, some form of constraint is required in the mobile bearing to prevent dislocation. The introduction of such constraint tends to, once again, create undesirable effects of wear and loosening. Various types of mechanical constraints have been proposed with varying degrees of success. For example, a pin and slot arrangement is disclosed in U.S. Pat. No. 6,203,576. Other arrangements such as T-shaped pins and dove tail slots are shown in Afriat, J., Larrouy, F., Is Dislocation A Specific Risk Inherent in Mobile-Bearing Total Knee Replacements?, (Published on the Internet and available at: http://www.maitrise-orthop.com/corpusmaitri/orthopaedic/88_afriat/afriatus.shtml). Such arrangements have not been completely successful because of continuing tendencies to dislocate and due to over constraining movement at the interface.

There have also been some attempts to develop applications that utilize non-mechanical forces to augment the treatment of particular orthopedic problems. For example, pulsating electromagnetic field has been used as an adjunct to stimulating bone healing. Biochemical and biomaterial means have been used to alter the milieu at fracture sites and in joints to aid healing and to decelerate disease processes. Others have attempted to utilize magnetic fields in treatment of bone and joint disorders as well. For example, U.S. Pat. No. 4,024,588 to Janssen, et al. describes artificial joints with magnets. U.S. Pat. No. 4,029,091 to Von Bezold et al. discloses a method of applying plates to fractured bones so as to allow limited motions of the bone fragments when subjected to an externally generated electromagnetic force. U.S. Pat. No. 4,322,037 to Esformes et al. suggests a elbow joint including mechanically interlocking joint components with the inclusion of a magnetic force on the joint. U.S. Pat. No. 5,595,563 to Moisdon discloses a method of repositioning body parts through magnetic induction generated by extra-corporeal magnetic or electromagnetic devices. Moisdon also discloses repositioning a degenerated joint to a pre-degenerated state such that the joint tissues can heal. In doing so, magnets are implanted into opposing joint bone portions and adjusted from the exterior of the body. The magnets are adjusted, external to the body, until their repulsive forces create the desired joint orientation. U.S. Pat. No. 5,879,386 to Jore describes an apparatus to hold bones apart which can also be adjustable from inside the joint, through arthroscopic means, for example. The disclosed devices and methods have limited uses for specific orthopedic problems. However, these designs are generally not practically feasible due to errors or misconceptions related to the practical application of orthopedic surgical treatments or, more importantly, a lack of understanding concerning the properties of permanent magnets in relationship to the mechanical environment found in the human body, especially as they relate to the normal functions of bones and joints.

More recent advancements in orthopaedic prosthesis are disclosed by the present applicant, Hyde, in U.S. Pat. No. 6,387,096. Hyde generally discloses the use of magnetic arrays in orthopaedic prosthesis. The disclosed array are arranged to provide composite magnetic fields for locating and confining motion. However, these designs generally require more complex magnet configurations that may not be suitable or required in all applications.

There therefore remains a need in the art for a prosthesis that adequately accommodates shear forces and simultaneously addresses problems such as excessive wear and prosthetic loosening.

### SUMMARY OF THE INVENTION

The present invention provides a prosthesis comprises a first component adapted to carry an articulating surface of a joint and a second component, adjacent the first component, configured and dimensioned to be fixed with respect to a bone of the joint. Further included are magnetic elements disposed in each of the first and second components, wherein the magnetic elements cooperate to control motion between the first and second components.

In an alternate embodiment, a prosthesis of the present invention comprises a first component adapted to carry an articulating surface of a joint and a second component, adjacent the first component, configured and dimensioned to be fixed with respect to a bone of the joint. Also included in this embodiment are physical stops configured and dimensioned around the second component near its perimeter. The stops protrude toward the adjacent first component such that planar movement of the first component with respect to the second component is constrained.

The present invention also includes a prosthesis that comprises a first component adapted for operative connection to a first bone portion and a second component adapted for operative connection to a second bone portion opposed to the first bone portion. Magnetic elements are disposed in each of the first and second components to interact with the element in the opposite component. The magnetic elements being configured in a grid pattern with a plurality of oppositely oriented magnetic polarity regions generated thereby. The magnetic elements cooperate to control motion between the first and second components and thus between the first and second bone portions.

A prosthesis according to the present invention comprises a first component adapted to carry an articulating surface of a joint and a second component, adjacent the first component, configured and dimensioned to be fixed with respect to a bone of the joint. Magnetic elements are disposed in each of the first and second components. The magnetic elements cooperate to control motion between the first and the second components. The magnetic elements are configured in a grid pattern such that a plurality of opposite oriented magnetic polarity regions are generated. Furthermore, the grid pattern is formed by interlacing a base unit comprised of multiple magnets with polarities oriented such that magnetic flux of the base unit is substantially concentrated on one surface of the base unit.

According to another embodiment a prosthesis comprises a first component adapted for operative connection to a first bone portion and a second component adapted for operative connection to a second bone portion opposed to the first bone portion. Soft magnetic material is disposed within the first component and hard magnetic material is disposed within the second component. The soft and hard magnetic material being positioned for magnetic attraction therebetween. Furthermore, at least one of the hard or soft magnetic materials has a center point and is configured and dimensioned to vary the magnetic attraction as the opposite magnetic material is moved away from the center point.

A prosthesis according to the present invention comprises a first component configured and dimensioned to carry an articulating surface of a joint and a second component configured and dimensioned to interface with the first component on one surface and receive a bone of a joint on an adjacent surface. First magnetic material is disposed within the first component and second magnetic material is disposed within the second component. The second magnetic material is concentrated substantially centrally and around a periphery such that a magnetic attractive gradient is formed between the first component and the second component when the first component translates with respect to the second component. Furthermore, the first magnetic material and the second magnetic material interact to maintain the first component in substantially an equilibrium position with respect to the second component.

### BRIEF DESCRIPTION OF THE FIGURES

FIG. 1A is an exploded, schematic view of an embodiment of a knee prosthesis with a magnetic coupler mobile bearing according to an embodiment of the present invention;

FIG. 1B is a schematic cross section of an alternative mobile bearing according to the present invention;

FIG. 1C is a perspective view of a further alternative mobile bearing according to the present invention;

FIG. 1D is a schematic view of another alternative embodiment of a mobile bearing according to the invention;

FIG. 2 is a schematic cross-section of an alternative embodiment of the invention, incorporating the embodiments of FIGS. 1A-1D together in a single device;

FIG. 3 is an exploded, schematic view another embodiment of a knee prosthesis with a magnetic coupler mobile bearing according to another embodiment of the present invention;

FIG. 4 is an exploded, schematic view an embodiment of a shoulder prosthesis with magnetic coupler mobile bearings according to an embodiment of the present invention;

FIG. 5 is a schematic cross-section showing an embodiment of a magnetic coupler mobile bearing for a prosthesis according to an embodiment of the present invention;

FIG. 6A is a plan view of a prosthetic component illustrating a distribution of magnetic material according to an embodiment of the present invention;

FIG. 6B is a plan view of another embodiment of the distribution of material of a magnetic coupler mobile bearing according to another embodiment of the present invention;

FIG. 6C is a plan view of yet another embodiment of the distribution of material of a magnetic coupler mobile bearing according to yet another embodiment of the present invention;

FIG. 7A is a schematic view of a further alternative embodiment of the present invention;

FIG. 7B is a schematic cross-section showing yet another embodiment of the magnetic coupler mobile bearing of the present invention;

FIGS. 8 and 9 are perspective views of a further preferred embodiment of a magnetic coupler mobile bearing according to a further embodiment of the present invention;

FIG. 10 is a plan view of an embodiment of a magnetic grid coupler of one component of a mobile bearing according to an embodiment of the present invention;

FIG. 11 is a schematic view of a knee prosthetic with a magnetic grid coupler mobile bearing of FIG. 9 incorporated therein;

FIG. 12 is schematic view of another embodiment of a mobile bearing according to another embodiment of the present invention;

FIG. 13 is a schematic view of yet another embodiment of a mobile bearing according to yet another embodiment of the present invention; and

FIG. 14 is still another embodiment of a mobile bearing magnetic coupler according to still another embodiment of the present invention.

### DETAILED DESCRIPTION OF THE INVENTION

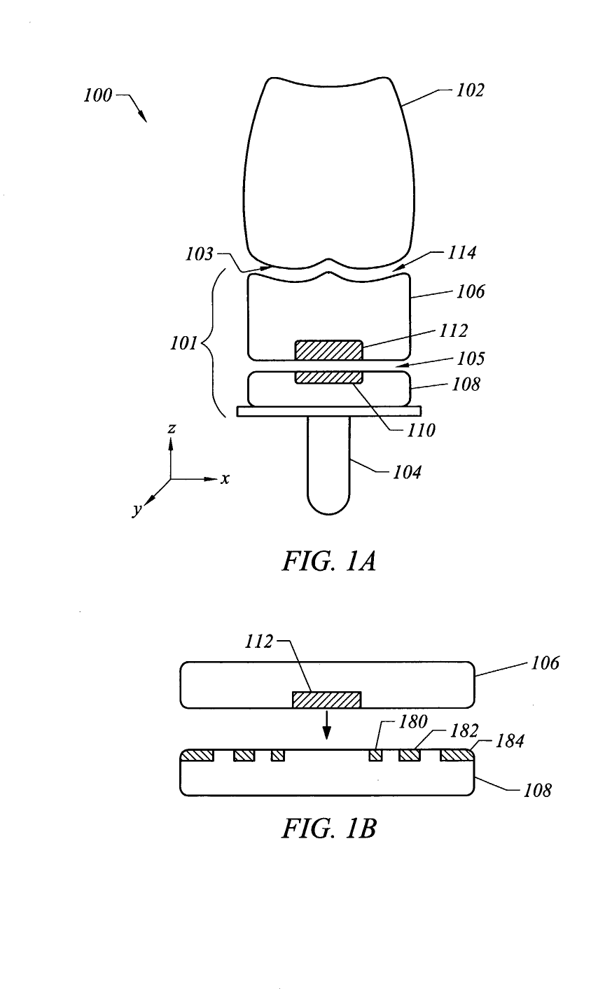

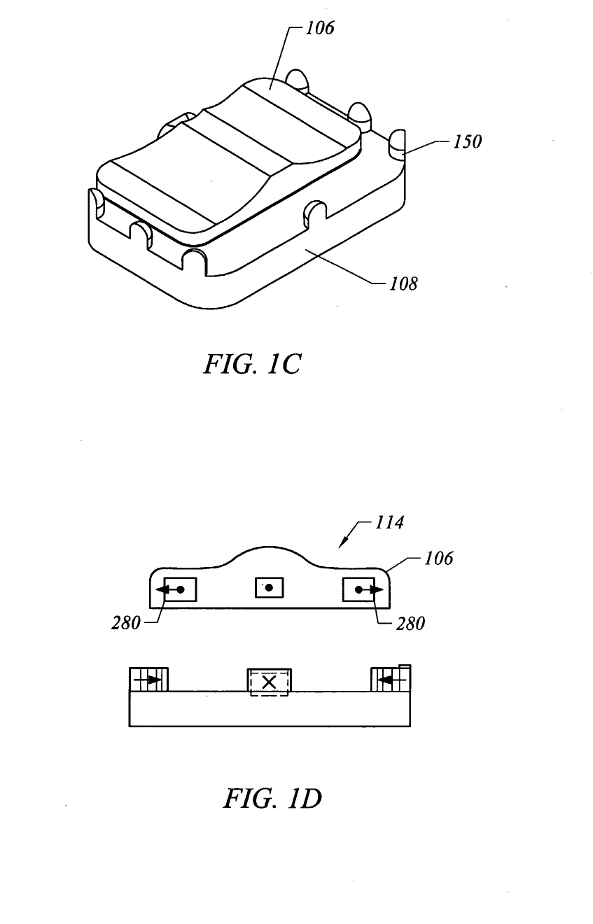

While FIG. 1A shows an orthopedic prosthesis 100 for the knee joint according to an embodiment of the present invention, it should be understood that the present invention is applicable to orthopedic prosthesis for any articular joint. Prosthesis 100 includes a first or femoral component 102 having an articular surface 103 and a second or tibial stem component 104. Mobile bearing 101 is disposed between the first and second components and comprises an articulation component 106 and a base component 108. The articulation component 106 and the base component 108 are preferably constructed from approved, implantable articulation materials, such as, ceramics, plastics, metals or, ultra high molecular weight polyethylene, for example. The articulation component 106 is configured with an articulation surface 114 that mates and articulates with articulation surface 103.

Prosthesis 100 may be provided as a complete prosthesis itself, or articulation component 106 and base component 108 may be configured to attach to existing tibial stem components 104 and femoral components 102 and articulate with existing or implanted prosthetic articulation surfaces, such as articulation surface 103. Suitable examples of knee prosthesis that articulation component 106 and base component 108 may be adapted to be utilized with is the AMK® made by DePuy Orthopaedics, Inc., Warsaw, Ind.; Ascent® made by Biomet, Inc., Warsaw, Indiana; Genesis II® made by Smith & Nephew, Inc., Memphis, Tenn.; or the like.

In a preferred embodiment, hard magnetic material 112 is embedded into the articulation component 106. Hard magnetic material 112 is positioned substantially opposite articulation surface 114 of articulation component 106. Preferably hard magnetic material 112 is entirely embedded or encased within the articulation component 106, such that no portion of the hard magnetic material is exposed. It is also preferred that articulation component 106 be a material that does not interfere with the magnetic flux produced by the hard magnetic material 112. As is understood by those skilled in the art, “hard magnetic material” refers to one or more permanent magnets, permanent magnets having composite structures or materials, or any suitable material that is capable of permanently sustaining a magnetic field.

In a preferred embodiment, base component 108 is configured to attach to, or be an integral part of typical orthopaedic components that attaches to a bone. In the embodiment of FIG. 1A, the base component 108 is configured to attach to a typical tibial stem component 104 that is configured to be implanted into the tibia of a patient. Embedded within base component 108 is soft magnetic material 110. As is understood by persons skilled in the art, “soft magnetic material” refers to any material, such as iron for example, that is not inherently magnetic, but is capable of sustaining a magnetic field when exposed to a permanent magnet. Collectively, cooperating magnetic materials, whether hard alone or hard and soft, are referred to herein as “magnetic elements.” Hard magnetic material 112 produces a magnetic flux that attracts the soft magnetic material 110 in the base component 108 to the hard magnetic material 112. Because articulation component 106 and base component 108 are coupled to each other only by magnetic flux of the hard magnetic material 112, the two components are capable of movement relative to each other, thus forming a mobile bearing 101 through the dynamic constraint of the magnetic flux of the hard magnetic material 112. Furthermore, since the articulation component 106 is moveable it will be urged by the magnetic flux to return to a preferred position with the base component 108 when it is displaced during knee function.

Mobile bearing 101 thus adds three, controlled degrees of freedom. Specifically, mobile bearing 101 enables the articulation component 106 to translate in the x-y plane and rotate about the z-axis (FIG. 1A) relative to the base component 108. Control is achieved through the constraint of the magnetic fields. As is understood in the magnetic arts, the interaction of the hard and soft magnetic material can be describes as forming a magnetic potential well between the two materials that acts to urge the materials to an equilibrium point, typically defined by a center of the potential well created by the overlapping two materials. In use, one component, such as the articulation component 106, can move within the confines of the magnetic potential well without overcoming the confines of the magnetic potential well. The kinetic energy being thus dissipated in the confines of the potential well. However, to move one component beyond the confines of the magnetic potential well, the cumulative magnetic interaction must be overcome. As will be appreciated by those skilled in the art, other forces such as friction and resistance of various tissues must be accounted for in the design of the prosthesis for each particular application. Thus, a person skilled in the art may craft various shapes, sizes, and combinations of hard and soft magnetic materials to control and manipulate motion between components 106 and 108 so as to minimize to the greatest extent possible wear between the components and mechanical loads transmitted to the supporting bone.

When the joint containing a prosthesis according to an embodiment of the present invention is articulated, mechanical stresses are generated between components 106 and 108. If these reach a magnitude greater than the potential energy associated with magnetic elements 110 and 112 and the friction, the two components will be displaced out of the potential well. In preferred embodiments, the forces associated with the magnetic elements are not so great, such that, following displacement, the articulation component 106 and the base component 108 can re-align to a substantially preferred position with normal muscular and ligamentous functions. In use, the muscles, tendons, ligaments and similar anatomical structure surrounding the joint apply a sufficient force to substantially re-align the articulation component 106 with the base component 108 following displacement. Preferably, the articulation component 106 may translate between about 0 and +/−9 mm on the x-y plane relative to the base component 108.

Articulation component 106 may also rotate about the z-axis (FIG. 1A) with respect to base component 108. In one preferred embodiment, articulation component 106 rotates about base component 108 without a static mechanical constraint. For example, there is no static non-magnetic stop to the rotation about the z-axis, which is preferably between about 0 and +/−90 degrees. Control of the rotation is enabled by the anatomical structures of the particular patient. The magnetic force of the hard magnetic material 112, attracting the soft magnetic material 110, creates a dynamic constraint against rotation similar to that which was described above with respect to translation of the components in the x-y plane. The articulation component 106 can rotate freely within the potential well formed by the hard and soft magnetic material. Following displacement of the components from forces generated during standing, walking, or the like, the natural joint realigning anatomical functions of the joint, such as the ligaments, and tendons, will re-align the articulation component 106 and the base component 108 following removal of the displacement force.

The movement between articulation component 106 and base component 108 disperses mechanical stresses and enables the articular surfaces of the artificial joint to, more likely, maintain proper alignment and an increased congruency with respect to each other during articulation. Increased congruency provides a larger surface contact area over which the mechanical stresses can be distributed, thereby, decreasing friction and wear of the prosthetic components.

FIG. 1A illustrates just one basic embodiment of the invention that comprises one of four basic “building blocks” for designing a customized prosthesis according to the present invention. FIGS. 1B-1D illustrate additional building block embodiments that also may be utilized alone or in any combination, including all together, in order to address particular needs. Persons skilled in the art will also recognize the virtually limitless variations that may be made to the basic building block embodiments, only a few examples of which may be reasonably discussed in detail herein, without departing from the scope of the invention. Also, a person of ordinary skill in the art will recognize that the hard magnetic material or the soft magnetic material can be incorporated into either the articulation component or the base component. Furthermore, a person of ordinary skill in the art will recognize that a mobile bearing of the present invention can consist of one or more levels of movement planes. An example of multiple levels of movement planes includes the articulation component include a base member and an insert member. The insert member being disposed on the base member. Therefore, in this example, the insert member is moveable on the base member while the articulation component is moveable on the base component. Inserting multiple mobile bearing movement planes within a joint decreases transmission of forces to the prosthesis-cement-bone interface. Also, relative thicknesses and surface areas of the magnetic materials may be advantageously manipulated by persons skilled in the art.

For arrangements of magnetic material pairs, if the ratio of their thickness is substantially less than one, the soft magnetic material has a tendency to be attracted to the edge of the hard magnetic material instead of the center. The smaller the thickness ratio, the greater the tendency will be for the soft magnetic material to be attracted to the edge. Likewise, for arrangements of magnetic material pairs, if the ratio of their surface area is substantially less than one, the soft magnetic material has a tendency to be attracted to the edge of the hard magnetic material instead of the center. The smaller the surface area ratio, the greater the tendency will be for the soft magnetic material to be attracted to the edge. These properties associated with variations in thickness and surface area can be utilized and incorporated in magnetic coupler designs to take advantage of these variations in the physics that are particular to the relative shape of the magnetic material when one element is much thinner or has a smaller surface area than the other element.

FIG. 1B shows base component 108 in cross section. Base component 108 includes a series of progressively larger zones of soft magnetic material 180, 182, 184 arranged outwardly from the center. These zones may comprise, for example, discrete spots or annular rings. Each zone in conjunction with the hard magnetic material defines a progressively larger magnetic potential well as one moves in an outward direction. Thus, such an arrangement offers an advantage of producing a gradient such that an increasingly larger force for eccentric movement is required and at the same time a decreasingly smaller force for movement back toward a central alignment.

In certain applications, given the particular anatomy of the subject joint and the patient condition, it may be sufficient to constrain motion simply by augmentation of the patient anatomy with one or more magnetic potential wells as described. In other applications, it may be desirable to augment the magnetic potential wells described above with a more definitive, structural stop. FIG. 1C illustrates one example of a preferred peripheral stop according to an embodiment of the invention. In this embodiment the base component 108 is configured with a plurality of peripheral posts 150 that provide a structural stop at the limit of travel. Posts 150 protrude beyond the surface of the base component 108 at or near its perimeter such that the articulation component 106 is located within a defined area or “footprint.” The articulation component 106 is free to translate in the x-y plane and rotate on the z-axis within the footprint of the base component 108 up to the position where an edge of the articulation component 106 encounters a post. Posts 150 can be sized and shaped the same or can have varying sizes or shapes depending on the forces anticipated to be applied at a particular location. Furthermore, the articulation component 106 can be shaped to correspond to the amount of rotation and translation that is desired in each particular direction. In a preferred embodiment, the articular component 106 can rotate, about the z-axis, without physical constraint between about −30 degrees and +30 degrees with respect to the base component 108, and can translate between about −10 and +10 mm in the x-y plane before engagement with the peripheral stop. Persons of ordinary skill in the art may utilize other types or designs for mechanical stops without departing from the invention. The stops can be constructed from a hard material, an elastic material, a viscoelastic material that dissipates some impact energy upon contact, or the like. Furthermore, the number, shape, and size of the stops can be arranged to increase the surface area for impact. Therefore, distributing the impact forces over a larger surface area minimizes the impact forces being concentrated at any one point and, thereby reducing mechanical loosening or other damage.

In the provision of a structural stop it may be desirable to further reduce impact force generated when the articulation component 106 contacts the stop. This may be accomplished by the inclusion of magnetic bumpers as shown in FIG. 1D. In this embodiment, structural magnetic stops 250 protrude from base component 108 forming a perimeter barrier past which articulation component 106 can not proceed. The structural magnetic stops 250 include hard magnetic material 270 disposed on or within a protruding structure such as posts 260. The protruding structure can be part of base component 108 or a housing that receives base component 108. The magnetic field of hard magnetic material 270, represented by the arrow, is directed inward, toward the center of the footprint within which articulation component 106 is free to translate a predetermined amount in the x-y plane and rotate in the z-axis. Articulation component 106 also includes hard magnetic material 280 at or near its perimeter. Hard magnetic material 280 of articulation component 106 is configured to present a repulsive magnetic field with respect to hard magnetic material 270 at stops 260. In use, the repulsive magnetic fields between the two sets of hard magnetic material act to dissipate the impact energy between the articulation component 106 and the stops 250 such that there is less stress on the components and lower transmission of shock to the bone. The shape and size of the magnetic fields may be adjusted for optimum performance in a particular application by a person skilled in the art through appropriate selection of hard magnetic material composition and configuration.

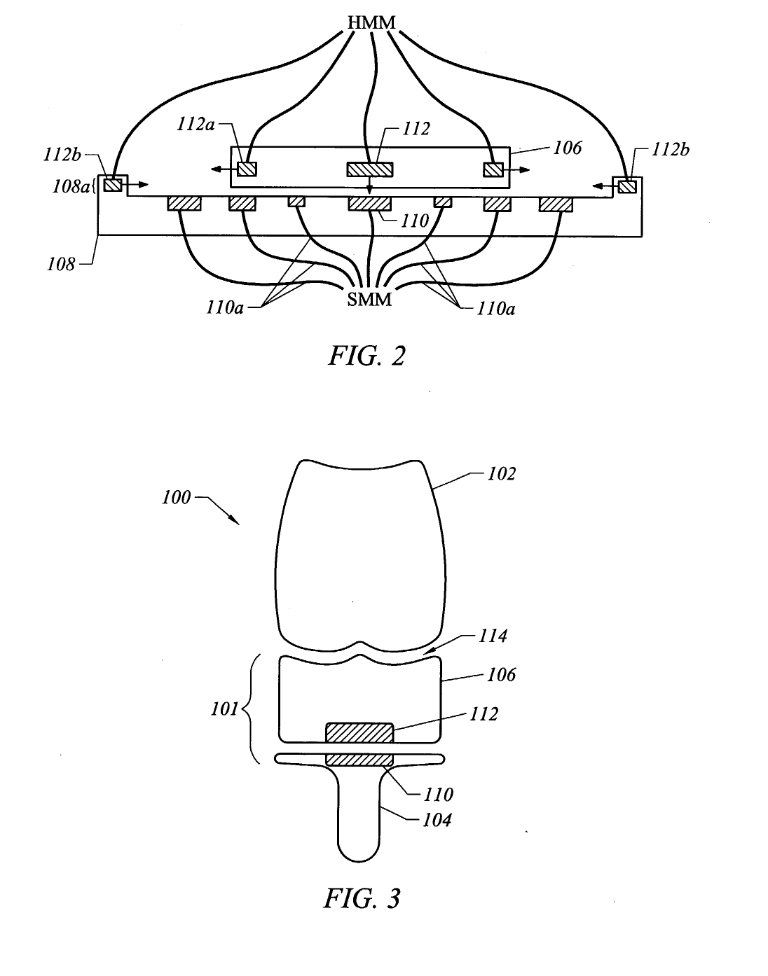

As previously mentioned, each of the above embodiments may be used alone or in various combinations. FIG. 2 illustrates one embodiment incorporating each of the embodiments of FIGS. 1A-1D. Articular component 106 includes hard magnetic material 112 at its center and further hard magnetic material 112a at the periphery. The peripheral hard magnetic material may be continuous or intermittent. Base component 108 includes central soft magnetic material 110 and patterned soft magnetic material 110a as described above. Structural stops 108a include hard magnetic material 112b arranged to repulse hard magnetic material 112a on articular component 106.

In an alternative embodiment as shown in FIG. 3, mobile bearing 101 is located between articular component 106 and tibial stem 104. Soft magnetic material 110 is embedded directly within tibial stem 104, for example in the tibial plate. This embodiment reduces the number of components in the prosthesis.

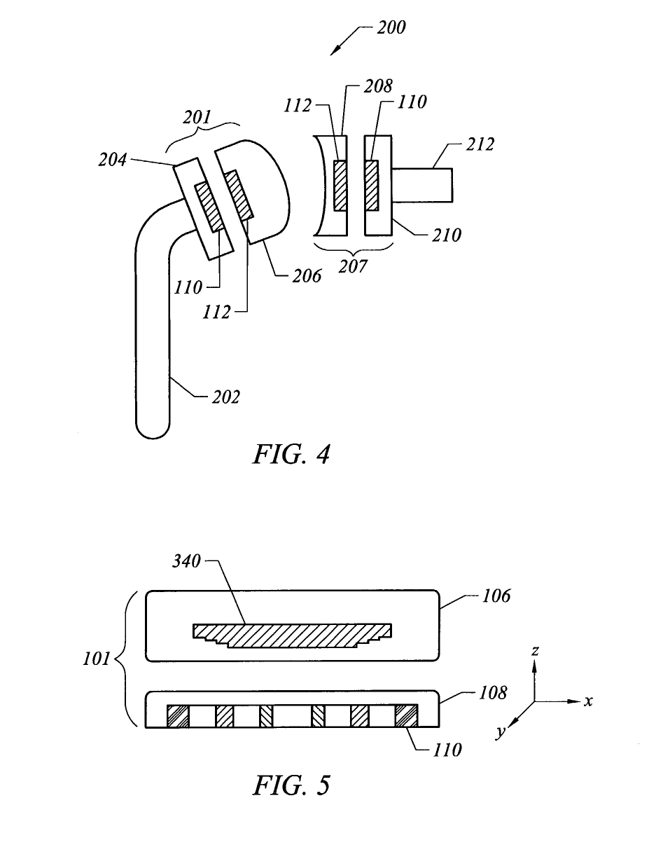

As will be appreciated by persons of ordinary skill in the art mobile bearings may be implemented at different levels and/or at multiple levels within a prosthetic component. In another alternative embodiment, shown in FIG. 4, prosthesis 200 is a prosthesis for a shoulder joint adapted with two magnetic coupler mobile bearings 201 and 207. The humeral stem 202 connects to humeral base component 204 with a conventional attachment, for example a Morse taper. Humeral base component 204 contains soft magnetic material 110 embedded therein. The humeral articulation component 206 is configured to articulate with the opposing articulation surface of the total or partial joint arthroplasty and is embedded with hard magnetic material 112. It is preferable that the hard magnetic material 112 be positioned to oppose the soft magnetic material 110 embedded within the base component. Thus, a mobile bearing 201, such as that described with respect to FIG. 1A, is established.

Second mobile bearing 207 may be provided for additional energy dissipation and to permit displacement in another plane. The glenoid articulation component 208 contains hard magnetic material 112. Adjacent to the glenoid articulation component 208 is the scapula base component 210. Embedded within the scapula base component 210 is soft magnetic material 110. Thus, mobile bearing 207 is established between the glenoid articulation component 208 and the scapula base component 210. In a preferred embodiment, the scapula base component 210 attaches to a bone stem component 212 by conventional attachment. FIG. 5 shows a further alternative embodiment of hard magnetic material 112 and soft magnetic material 110 embedded within articulation components 106 and base components 108, respectively. For simplicity sake, the articulation and base components are depicted as not joint specific and can be adapted for placement at any level or multiple levels in any joint replacement.

As shown in FIG. 5, hard magnetic material 112 is embedded within the articulation component 106. Preferably, hard magnetic material 112 is a solid unit comprising a continuous permanent magnet, such that a shaped uniform magnetic flux is produced. Once again, embedded within base component 108 is soft magnetic material 110.

In this embodiment, soft magnetic material 110 is preferably dispersed in an annular manner substantially located around the perimeter of the base component 108. This creates a footprint in the central region in which there is little or no attractive force between hard magnetic material 112 in articulation component 106 and soft magnetic material 110 in base component 108. As long as hard magnetic material 112 is located within this foot print, articulation component 106 can move relatively freely. However, a gradient of increasing force is created as the articulation component 106 moves substantially beyond the foot print. As hard magnetic material 112 moves closer to alignment with soft magnetic material 110, or toward the edge of the foot print, the magnetic force attracts the two components and gradually reduces or stops the movement of the articulation component106.

In a further alternative embodiment also illustrated in FIG. 5, hard magnetic material 112 is configured in a shaped or step manner, such that the magnet surface is closer to soft magnetic material 110 at some locations and spaced farther away in other locations. The portions of the magnet that are closer, in the z-axis direction, to soft magnetic material 110 will have a stronger magnetic attraction than the portions that are farther away from soft magnetic material 110. Therefore, a dynamic constraint gradient is formed in the coupler mobile bearing 101 of this embodiment.

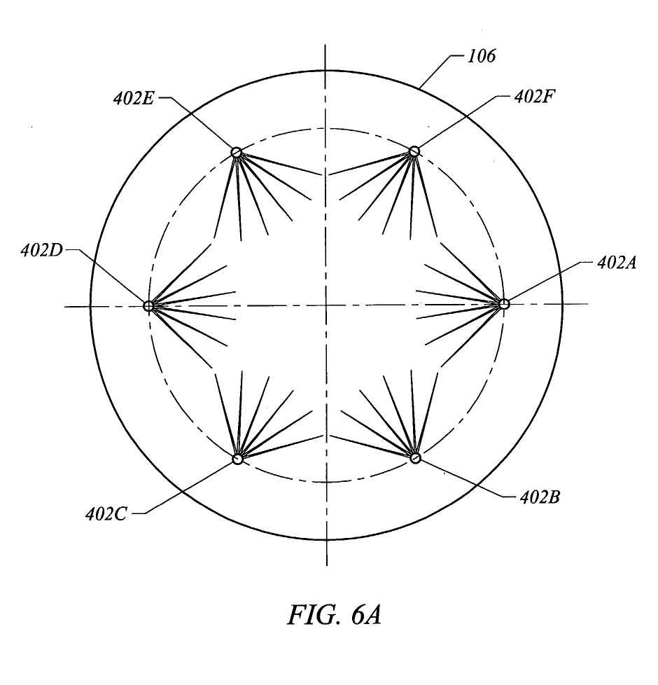

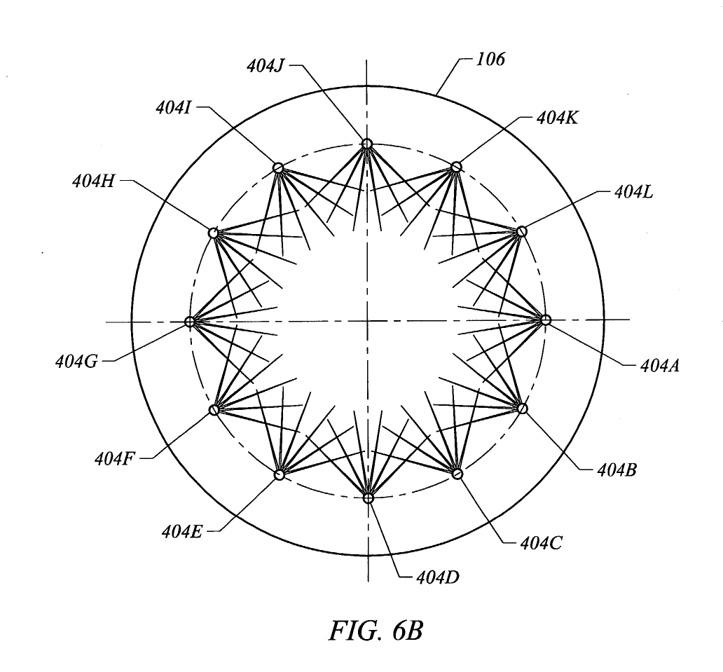

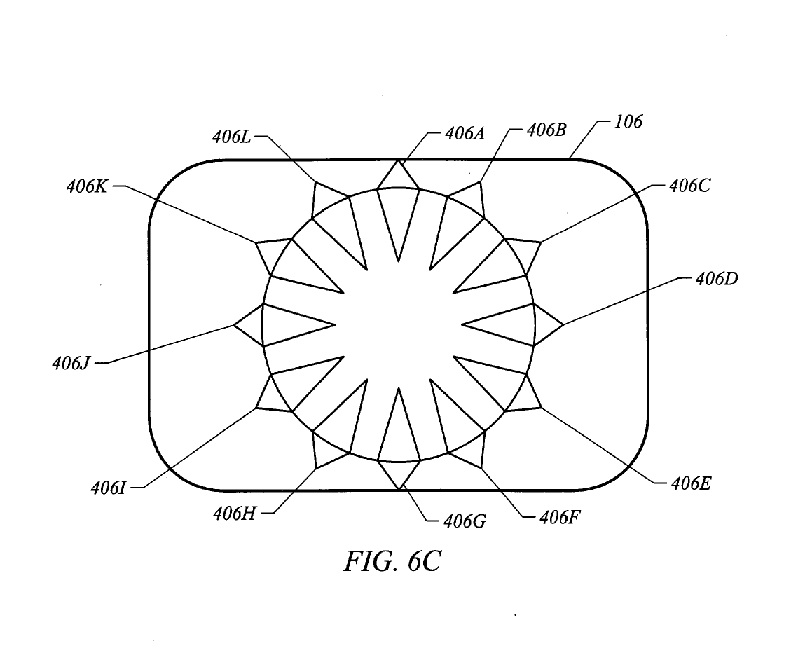

In order to provide a gradient in magnetic force over the range of displacement of the articular and base components, the hard or soft magnetic material may be distributed in a wide variety of patterns. FIG. 6A shows a plan view of one such distribution of hard magnetic material 402A-402F embedded within the articulation component 106. The pattern of material 402A-402F affects the relative amounts of overlapping surface areas between hard and soft magnetic material. The strength of magnetic attraction is proportional to that overlapping surface area. Therefore, the strength of the dynamic constraint between articulation component 106 and base component 108 changes as articulation component 106 moves relative to base component 108. In alternative embodiments, the gradient can be linear, non-linear, symmetric, or non- symmetric. As previously mentioned, in one embodiment, the hard magnetic material is dispersed in a pattern that is symmetric or asymmetric and the soft magnetic material 110 is a solid unit. In an alternative embodiment, the soft magnetic material 110 is arranged in a distribution as described above and the hard magnetic material 112 is configured as a solid unit. In a further alternative, both hard and soft magnetic materials may be provided in a desired distribution pattern.

FIG. 6B illustrates a further alternative embodiment in which the magnetic material is further overlapped. The further overlapping of material 404A-404L embedded within the articulation component 106 creates a greater magnetic force and thereby a stronger dynamic constraint gradient between the respective articulation component 106 and base component (not shown). In use, the amount of overlapping material can be adjusted to substantially match the magnitude and direction of forces to be absorbed into the system.

It is stressed that in preferred alternative embodiments, hard magnetic materials 112 may be constrained to a single solid unit and soft magnetic material 110 is varied in size, shape, and configuration according to the above descriptions, and the like. In use, varying the soft magnetic material 110 will produce the same resulting dynamic constraint mobile bearing as that described above. It is further stressed that soft magnetic material 110 of the base component 108 can be substituted with hard magnetic material, such that hard magnetic materials 112 of the articulation component 106 spatially oppose and magnetically attract or repel the hard magnetic material in the base component 108. In use, this forms a dynamic constraint mobile bearing system equivalent to the systems described above.

FIG. 6C shows an alternative embodiment to the overlapping magnetic material of FIGS. 6A and 6B. In FIG. 6C there is material 406A-406L embedded within the articulation component 106. As will be appreciated by persons ordinarily skilled in the art, material 406A-406L can be either hard magnetic material or soft magnetic material and can oppose either solid units or symmetric or asymmetric patterns of hard or soft magnetic material in the opposing component of the mobile bearing. Material 406A-406L primarily increases in cross sectional area as the distance increases away from the center of the articulation component 106. The base component 108 (not shown) contains either hard magnetic material or soft magnetic material. Therefore, as the articulation component 106 translates relative to the base component (not shown) the attractive force of the magnetic material 406A-406L increases corresponding to the increasing surface area overlap between the magnetic material 406A-406L of the articulation component 1-6 and the hard or soft magnetic material in the base component (not shown). This forms an increasing gradient of attractive magnetic force the further the hard or soft magnetic material of the base component (not shown) moves from the center of the articulation component 106. Therefore, the components are encouraged by the increasing magnetic gradient to remain within a preferred region. It is preferred that the maximum attractive magnetic force generated by the corresponding maximum cross sectional surface area overlap not exceed the forces of the anatomical structure of the surrounding joint. Therefore, when the components become maximally displaced the anatomical structures of the joint are capable of creating a force great enough to re-position the components to the preferred orientation.

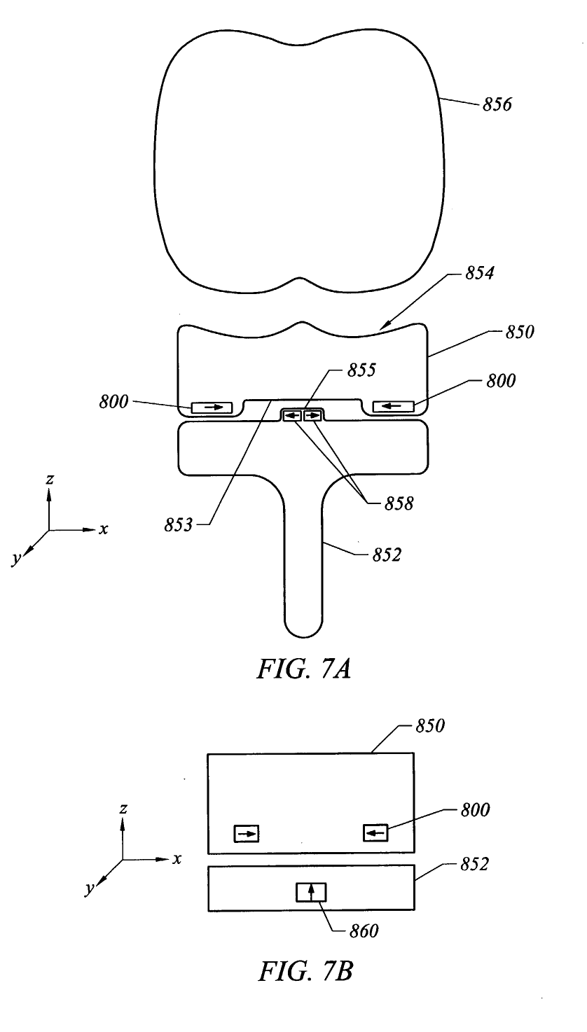

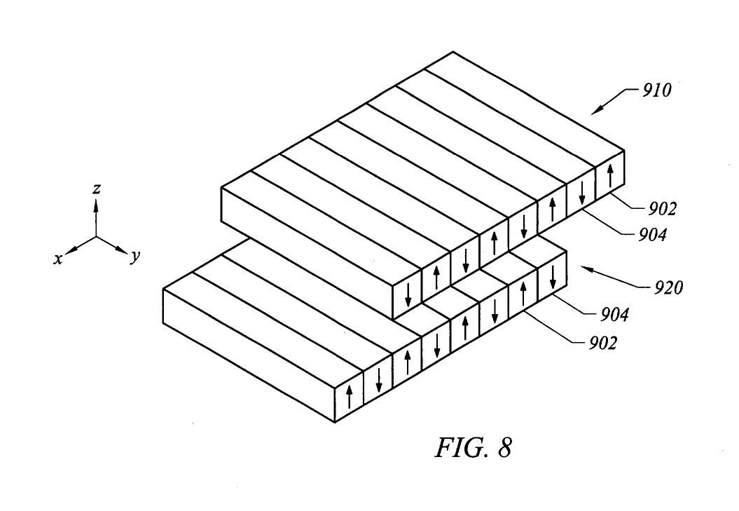

In a further alternative embodiment of the present invention, the hard magnetic material may be configured as a ring, e.g., ring array magnet 800 (opposite sides of which are shown in cross section in FIG. 7A). Articular component 850 provides articular surface 854, which cooperates with femoral component 856, and encompasses ring array magnet 800. Articular component 850 also defines recessed portion 853. In the embodiment as illustrated, the base component and tibial attachment component are combined in a single, tibial component 852. As will be appreciated, these components may be separately provided as described above. Locator pin 855 extends from tibial component 852 and is received in recessed portion 853 of the articular component. Preferably, locator pin 855 includes hard magnetic material 858 with polarity directed radially outward away from locator pin 855. Ring array magnet 800 is preferably configured such that the magnetic flux is oriented towards the center of the ring. Ring array magnet 800 and the hard magnetic material 858 embedded within locator pin 855 thus preferably cooperate such that the magnetic flux emanating from the ring opposes the magnetic flux emanating from the hard magnetic material 858 embedded within locator pin 855. Therefore, the articulation component 850 moves freely in the medial and lateral directions up to the point where the opposing magnetic fluxes begin to interact. It is preferred that the locator pin be rounded in shape with a long axis greater than about 10 millimeters in diameter and less that 80 percent of the surface area of the mobile bearing. The receiving hole for the locator pin is preferred to be roughly 3 millimeters larger than the diameter of the locator pin. It is more preferred that the locator pin be elliptical in shape with the larger radius of curvature be positioned to intercept the direction of movement carrying the largest energy loads. For example, in the knee joint the larger radius of curvature of an elliptical locator pin are positioned toward the anterior and posterior. This provides a larger surface area for contact between the component and the locator pin which in turn reduces the stresses at any given point, thereby, reducing mechanical failure.

Upon the application of a force that causes the articulation component 850 to move with respect to the tibial component 852, the magnetic fluxes disperse momentum of the motion and gradually retards the motion between the tibial component 852 and the articulation component 850. In use, this relieves the joint articulation surface of the prosthesis of forces that typically induce wear debris, mechanical loosening, material deformation of the articulation surfaces, and the like. Furthermore, because the magnetic fluxes of the ring 800 and the hard magnetic material 858 embedded within locator pin 855 oppose each other, the articulation component 850 is magnetically assisted in repositioning to a preferred position with respect to the tibial component 852 following displacement by a force. Locator pin 855 also may be configured to provide a mechanical stop at the desired limit of travel by abutting against the edge of recessed portion 853.

It will be appreciated by persons of ordinary skill in the art that hard or soft magnetic material may be incorporated into tibial component 852 to interact with ring magnet 800 in a manner as described above in connection with the various embodiments. Such additional magnetic elements will further increase the stability of the device as compared to prior art pin devices. Furthermore, the locator pin 855 may be replaced with a magnetic field emanating from hard magnetic material 860 embedded flush or recessed within tibial component 852, FIG. 7B. In this embodiment, the ring array magnet 800 is prevented from translating too far in any direction on the x-y plane by repulsion from the magnetic field emanating from the material embedded within the tibial component 852.

In yet another embodiment of the present invention, as shown in FIG. 8, hard magnetic material embedded within the prosthesis may be configured with regions of altering magnetic polarity. For example, upper magnetic unit 910 is embedded within one component of a prosthesis, such as, the articulation component 106 (e.g., FIG. 1A) and lower magnetic unit 920 is embedded within another portion of the prosthesis, such as, the tibial component, thereby forming a magnetic coupler mobile bearing as previously described. In one embodiment, hard magnetic material 902 is positioned opposite and adjacent to hard magnetic material 904. The polarity of the adjacent hard magnetic material 902 and 904 alters from the north pole of the hard magnetic material being oriented in a negative z-axis direction to the north pole of the magnet being oriented in a positive z-axis direction, respectively, as shown in the figure. The adjacent magnets thus create alternating magnetic potential wells and potential hills. When magnet 902 of the upper magnetic unit 910 is aligned with the corresponding magnet 902 of the lower magnetic unit 920, the upper and lower magnetic units 910 and 920 are attracted.

When a force exceeds the combined magnetic attractive force, the upper and lower magnetic units 910 and 920 can translate with respect to each other. Upon translation in the x-axis direction of either the upper or the lower magnetic units 910 or 920 with respect to the other magnetic unit, the attractive magnetic forces of the upper and lower magnetic units 910 and 920 become weakened. The amount of attractive force corresponds to the amount of overlapping surface area between the magnets 902 and 904 of the upper magnetic unit 910 with magnets 902 and 904 of the lower magnetic unit 920. During translation, when magnet 902 opposes magnet 904, the magnets repel each other and the upper magnetic unit 910 repels the lower magnetic unit 920. In this position the magnetic coupler 901 is unstable and the upper and lower magnetic units 910 and 920 will move to the next attractive orientation in the direction of the force. The units cycle as they move in the x-axis direction from attractive to repulsive configurations. This is equivalent to deepening the potential wells of the attractive configurations. This attractive/repulsive configuration of the magnetic elements dissipates the momentum by introducing an alternate wave of potential well to potential hills, introducing a form of an alternating braking mechanism. Furthermore, the magnetic material 902 and 904 can have different intrinsic strengths or shapes to generate a functional end pattern applicable to the particular location or patients requirements. Therefore, the magnetic coupler 901 absorbs and dissipates energy input into the prosthesis, such as shear forces, as the upper magnetic unit 910 moves relative to the lower magnetic unit 920.

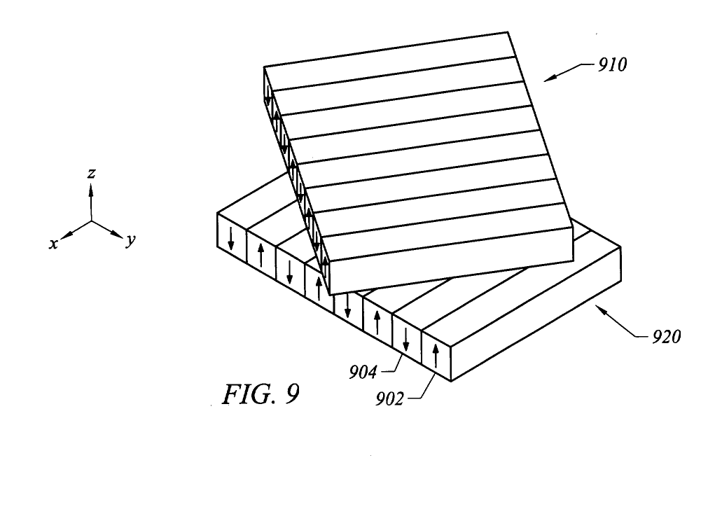

The upper magnetic unit 910 can rotate about the z-axis and with respect to the lower magnetic unit 920, as illustrated in FIG. 9. (FIG. 9 also shows upper and lower magnetic units with a larger number of adjacent bar magnets 902 and 904). Upon rotation, the force of magnetic attraction between the upper and lower magnetic units 910 and 920 corresponds to the amount of overlapping surface area of the attractive and repellant linear hard magnetic material 902 and 904 effectively making the potential well less steep. However, the attractive magnetic force attempts to re-orient the upper and lower magnetic units 910 and 920 to the orientation corresponding to the strongest attractive magnetic force between the magnetic units 910 and 920. Therefore, the magnetic coupler 901 disperses rotational energy about the z-axis into the magnetic flux of the magnetic units 910 and 920. The arrangement dissipates force energy that typically leads to destructive events within a joint prosthesis. Furthermore, the magnetic coupler 901 allows the articulation surfaces of the joint prosthesis to remain congruent following a rotational input force because the magnetic coupler 901 dissipates the rotational force, rotates, and returns to the orientation of greatest magnetic attraction when the rotational force is removed.

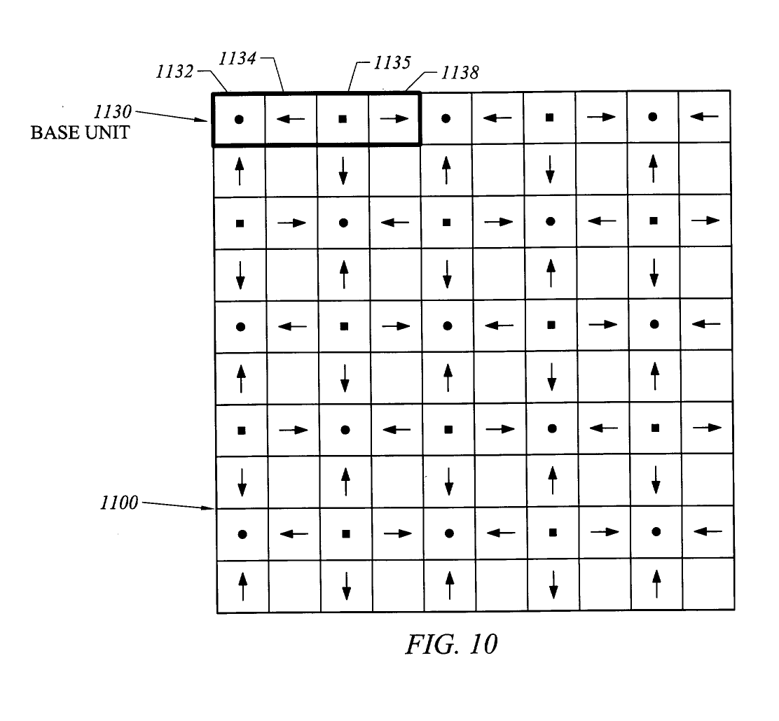

In another alternative embodiment, hard magnetic materials 112 of mobile bearing 101 comprise multiple hard magnetic material magnets coupled together in a grid pattern 1100, also referred to as a unit of a stepper 1100, as shown in FIG. 10. Within the grid pattern 1100 there are regions of north and south oriented polarities. An alternating field of magnetic potential wells and hills is thus formed effectively deepening the potential wells. A pair of matching grid patterns 1100 positioned opposite each other forms a stepper type mobile bearing 101. Portions of the grid 1100 produce a region of northern oriented polarity and other portions generate a southern oriented polarity. Repetitive base unit 1130 is composed of individual magnets arranged such that the magnetic polarity of each magnet is oriented in directions that cause the magnetic flux to add and concentrate on one surface of the repetitive base unit 1130, while making the field substantially zero on the opposite side. This effectively doubles the magnetic field strength for the same given mass of hard magnetic material.

In the embodiment illustrated in FIG. 10, repetitive base unit 1130 is generally composed of four magnets 1132, 1134, 1136 and 1138. The magnets in the base units 1130 oriented along the x-axis direction have magnetic polarities where magnet 1132 is oriented in the positive z-axis direction and the magnetic polarity of magnet 1134 is oriented in the negative x-axis direction, whereas the magnetic polarity of magnet 1136 is oriented in the negative z-axis direction and the magnetic polarity of magnet 1138 is oriented in the positive x-axis direction. Multiple base units 1130 are aligned linearly along the x-axis direction and also along the y-axis direction. The linear alignment of the multiple base units 1130 cross or overlap each other. Magnet 1132 abuts magnet 1138 of another base unit 1130, and so on. Also, according to the embodiment shown in FIG. 10, the multiple base units 1130 are interlaced by co-using magnet 1136 in both the x-axis and y-axis oriented base units 1130. By interlacing or overlapping magnets 1136 of both axis oriented base units 1130 the grid pattern shown in FIG. 10 is formed. Within the grid pattern 1100 there are spaces 1140 that are not occupied by magnets. It is preferable that the spaces 1140 are composed of non-magnetic material such as ceramic, metal, or plastic, a suitable example being ultra high molecular weight polyethylene, or the like. In an alternative embodiment the non-magnetic material may be substituted with a ferrous material that is suitable for implantation into the body.

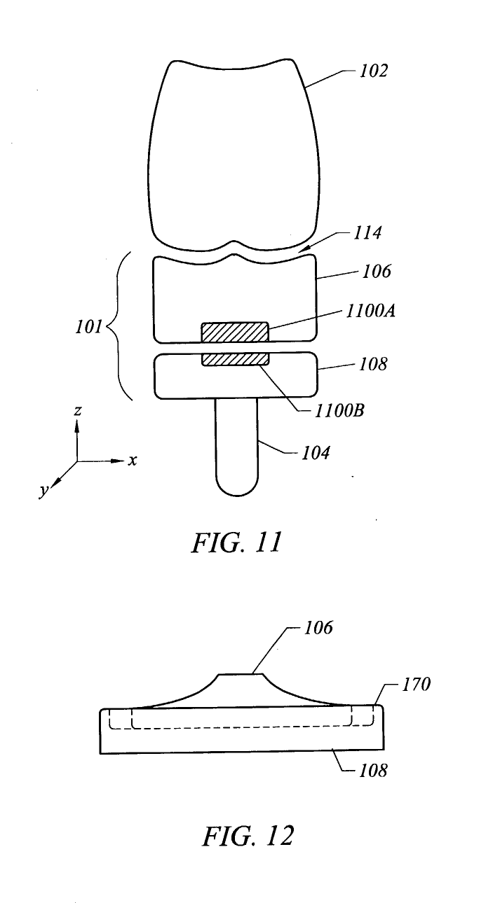

In use, a first grid 1100A is embedded within articulation component 106 and a second matching grid 1100B is embedded within base component 108, for example as shown in FIG. 11. The opposing first and second grids, 1100A and 1100B, attract and magnetically bind to one another when magnet 1136 is aligned with magnet 1132 of the opposing grid. The articulation component 106 translates in the x-y plane and can rotate about the z-axis with respect to the base unit 108 when an force is applied to the mobile bearing 101. When a force is applied to one component, the component moves as the magnets move within their potential wells. However, the components remain relatively stationary with respect to one another until the force applied is greater than the cumulative potential well force.

When the applied force is greater than the cumulative potential well force or attractive magnetic force of magnets 1136 with magnets 1132 the first grid 1100A moves and the articulation component 106 moves with respect to the base component 108. The components 106 and 108 then become unstable as the repulsive force of magnets 1136 in the first grid 1100A repel corresponding magnets 1136 in the second grid 1100B. The components then move to a more stable location where the magnetic forces bind the components 106 and 108 together. Therefore, magnets 1136 bind to the next sequentially placed magnets 1132 in the direction of the applied force. This can also be conceptualized as the grid 1100A repositioning to a different potential well in response to an applied force that is great enough to overcome the potential well the grid 1100A was positioned within.

The magnitude of the force applied to the system and the overall strength of the magnetically bound steppers through the magnitude and quantity of magnets 1132, 1134, 1136, and 1138 will determine how much movement is attained between components 106 and 108 in response to a given force. The binding, dislodging, and binding dissipates force input into the mobile bearing 101 system. It is preferred that following the removal of the force, the combined magnetic binding force of magnets 1136 with magnets 1132 is not so great that the anatomical features of the respective joint cannot realign the first and second grids 1100A and 1100B and therefore the articulation component 106 with the base component 108 to an equilibrium position.

In a further alternative embodiment the second grid 1100B is replaced with ferrous or other magnetic material that corresponds to the spacing of the attractive magnetic flux of the first grid 1100A. In use, the magnetic material is magnetically bound to the first grid 1100A at the attractive magnetic flux regions, thereby forming a mobile bearing magnetic coupler.

It is stressed that the stepper can be of many variable configurations. The stepper pattern 1100 of FIG. 10 is meant to be an example and not exhaustive of the possible embodiments. The spacing between the attractive and repulsive regions of the stepper pattern and the strength of the magnetic binding potential may be altered to acquire the desired relative motion within the mobile bearing for the particular application, prosthetic, joint, and the like the mobile bearing is incorporated into.

FIG. 12 illustrates a further variation of the structural stop described above. In this embodiment perimeter barrier stop 170 fully surrounds the articulation component 106. Perimeter barrier stop 170 can be an extension of the base component 108 or it can be a separate component that houses the base component 108. In a preferred embodiment the surfaces of the components 106 and 108 that interact with one another are highly polished surfaces 106A and 108B, respectfully. The articulation component 106 can translate in the x-y plane and rotate around the z-axis a predetermined amount depending on the tolerance between the perimeter of the articulation component 106 and the perimeter barrier stop 170. This embodiment provides even greater contact area at the edges to increase dissipation of loads.

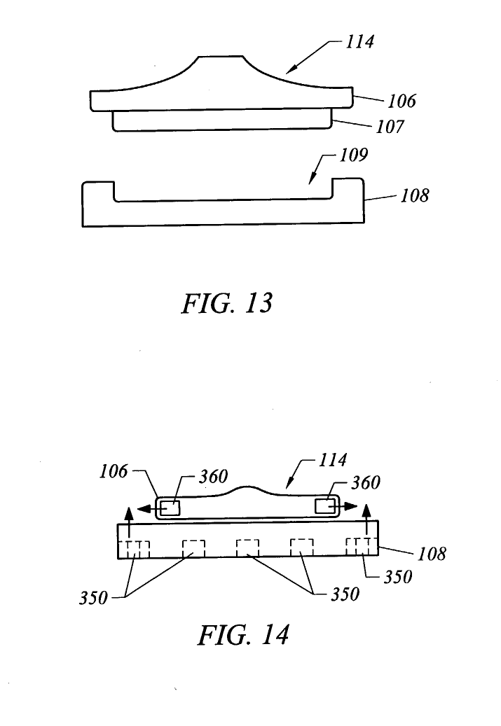

FIG. 13 shows a further alternative embodiment of the mobile bearing 101 of the present invention where the movement between the articulation component 106 and the base component 108 is limited to particular x-y plane translation and rotation around the z-axis. The surface of the articulation component that is opposite the articulation surface 114 protrudes and is received by a recess in the mating surface of the base component 108. The articulation component 106 can translate in the x-y plane and rotate in the z-axis a predetermined amount depending on the tolerance between the protrusion 107 and the recess 109.

FIG. 14 illustrates another embodiment in which base component 108 of mobile bearing 101 is configured with hard magnetic material 350 oriented around its perimeter. Hard magnetic material 350 is completely encased within the base component 108 and configured with its magnetic field represented by the arrow. The magnetic field forms a perimeter barrier defining a footprint where the articulation component 106 is free to translate in the x-y plane and rotate around the z-axis. The articulation component 106 contains hard magnetic material 360 around or near its perimeter. The hard magnetic material 360 encased within the articulation component 106 has its magnetic field directed, as shown by the arrow, toward the perimeter of the base component 108. As the articulation component 106 translates in the x-y plane and rotates around the z-axis the translation and rotation is dampened and preferably dissipated when the opposing magnetic fields intersect, urging the mobile bearing to an equilibrium position. This creates a magnetic perimeter barrier past which the articulation component 106 cannot pass.

It will be understood by persons skilled in the art that many different configurations of magnets and magnetic material may be used in connection with the present invention. For example, various magnetic arrays as described in parent application Ser. No. 09/849,227, which incorporated by reference, may also be used. In general, persons of skill in the art will recognize that the exemplary embodiments described herein are merely illustrative of the principles of the invention, as it is not possible to economically describe every conceivable combination of magnets and/or magnetic material suitable for use in the present invention.

What is claimed is:

1. A prosthesis, comprising:
a first component adapted to carry an articulating surface of a joint;
a second component, spaced from the articulating surface by the first component, configured and dimensioned to be fixed with respect to a bone of the joint; and
magnetic elements disposed in each of said first and second components, wherein said elements cooperate to control motion between said first and second components.

2. The prosthesis of claim 1, wherein said components contact each other on contact surfaces, the first component contact surface being disposed opposite a joint articular surface and the second component contact surface being disposed opposite a bone fixation point of the prosthesis.

3. The prosthesis of claim 2, wherein said contact surfaces are polished thereby establishing a low friction surface.

4. The prosthesis of claim 1, wherein said magnetic elements comprise a hard magnetic material disposed in one said component and at least one of another a hard magnetic material or a soft magnetic material disposed in the other component.

5. The prosthesis of claim 1, wherein said second component is configured to couple between said first component and a bone attachment component that is fixed to a bone of the joint.

6. The prosthesis of claim 1, wherein said first and said second components are made from a biocompatible material.

7. The prosthesis of claim 1, further comprising at least one physical stop configured and dimensioned on said second component near its perimeter, wherein said at least one physical stop protrudes toward said adjacent first component such that planar translation and rotation of said first component with respect to said second component is predictably constrained.

8. The prosthesis of claim 7, wherein said physical stop is configured and dimensioned with a substantially planar contact surface.

9. The prosthesis of claim 7, wherein said physical stop comprises a substantially elliptical protrusion from one of said components with a long axis of at least 10 millimeters but encompassing not more than; 80 percent of a surface area of the second component and wherein there is about a 3 millimeter gap between said substantially elliptical protrusion and said other component such that each of said components can move with respect to each other.

10. The prosthesis of claim 7, wherein said physical stop is disposed with magnetic elements configured and dimensioned to interact with corresponding opposing magnetic stop elements configured and dimensioned and disposed within said first component.

11. The prosthesis of claim 1, further comprising at least one stop wherein said stop is a magnetic field emanating from at least one magnetic element subsurfacely disposed within one said component and opposing a magnetic field emanating from at least one magnetic element subsurfacely disposed within the other component.

12. The prosthesis of claim 1, wherein said magnetic elements are encased within said first and said second components such that said magnetic elements are maintained separate from contact with body fluids and components.

13. The prosthesis of claim 1, wherein said magnetic elements comprise substantially disc like magnets disposed substantially near a center of the components and substantially aligned with one another.

14. The prosthesis of claim 1, wherein said magnetic elements comprise at least one series of progressively larger zones of magnetic elements disposed within one said component and a simple substantially solid shaped magnet disposed within the other said component.

15. The prosthesis of claim 1, wherein:
said first component contains a substantially solid shaped magnetic element positioned substantially within a center of said first component; and
said second component contains a substantially centered magnetic element and a plurality of substantially peripherally positioned magnetic elements configured and dimensioned such that as the first component moves toward a periphery of the second component, the components are increasingly attracted by an increasing magnetic gradient.

16. The prosthesis of claim 1, wherein said magnetic elements comprise a grid of magnetic elements configured with a plurality of oppositely oriented magnetic polar regions.

17. The prosthesis of claim 1, wherein said magnetic elements comprise a plurality of linear hard magnetic material configured adjacent each other with alternating polarities.

18. The prosthesis of claim 1, further comprising:
at least one further component configured and dimensioned to be positioned between said first component and said second component;
wherein said at least one further component is disposed with further magnetic elements; and
wherein said further magnetic elements interact cooperatively with said magnetic elements in said first component and said second component to further control motion between all said components.

19. The prosthesis of claim 1, wherein said first component comprises a base member and an insert member disposed on the base member.

20. The prosthesis of claim 19, wherein said insert member is moveable with respect to the base member.

21. The prosthesis of claim 20 further comprising additional magnetic elements disposed in each of said base member and insert member and cooperating to control relative motion between said members.

22. A prosthesis, comprising:
a first component adapted to carry an articulating surface of a joint;
a second component, adjacent the first component, configured and dimensioned to be fixed with respect to a bone of the joint; and
physical stops configured and dimensioned around said second component near its perimeter, wherein said stops protrude toward said adjacent first component such that planar movement of said first component with respect to said second component is constrained.

23. The prosthesis of claim 22, wherein said physical stops are configured and dimensioned with a substantially planar contact surface such that during contact with said first component mechanical stresses are distributed over said substantially planar contact surface.

24. The prosthesis of claim 22, wherein said physical stops form a continual perimeter substantially encircling said components.

25. The prosthesis of claim 22, wherein said physical stops is a separate component configured and dimensioned to receive a component around its perimeter and provide a physical barrier for the other component.

26. The prosthesis of claim 22, wherein said physical stop is a substantially elliptical protrusion from one said component having a long axis of at least about 10 millimeters but encompassing not more than 80 percent of a surface area of the second component and wherein there is about a 3 millimeter gap between said substantially elliptical protrusion and the other component such that the components can controllably translate and rotate with respect to each other.

27. The prosthesis of claim 22, wherein said physical stops are disposed with magnetic elements configured and dimensioned to interact with corresponding opposing magnetic stop elements configured and dimensioned and disposed within said first component.

28. The prosthesis of claim 22, further comprising at least one stop wherein said stop is a magnetic field emanating from a magnetic element subsurfacely disposed substantially within one said component and opposing a magnetic field emanating from magnetic elements substantially embedded within the other said component.

29. A prosthesis, comprising:
a first component adapted for operative connection to a first bone portion;
a second component adapted for operative connection to a second bone portion opposed to the first bone portion; and
magnetic elements disposed in each of said first and second components to interact with the element in the opposite component, said magnetic elements being configured in a grid pattern with a plurality of oppositely oriented magnetic polarity regions generated thereby, wherein said magnetic elements cooperate to control motion between said first and second components thus between said first and second bone portions.

30. The prosthesis of claim 29, wherein said first component is operatively connected to the first bone portion across an articulating joint surface.

31. The prosthesis of claim 29, wherein said grid pattern is formed by a repetitive pattern comprising a repeated base unit, wherein said base unit comprises plural magnets configured and dimensioned with polarities oriented to substantially concentrate magnetic flux of the base unit on one surface thereof.

32. The prosthesis of claim 31 wherein said base unit comprises four magnets coupled together each having an associated magnetic polarity oriented differently from the other magnets in the base unit.

33. A prosthesis comprising:
a first component adapted to carry an articulating surface of a joint;
a second component, adjacent the first component, configured and dimensioned to be fixed with respect to a bone of the joint;
magnetic elements disposed in each of said first and second components, wherein said magnetic elements cooperate to control motion between said first and said second components;
wherein said magnetic elements are configured in a grid pattern such that a plurality of opposite oriented magnetic polarity regions are generated; and
wherein said grid pattern is formed by interlacing a base unit comprised of multiple magnets with polarities oriented such that magnetic flux of said base unit is substantially concentrated on one surface of said base unit.

34. A prosthesis, comprising:
a first component adapted for operative connection to a first bone portion;
a second component adapted for operative connection to a second bone portion opposed to the first bone portion;
a soft magnetic material disposed with said first component; and
a hard magnetic material disposed with said second component, said elements being positioned for magnetic attraction therebetween;
wherein at least one of said hard or soft magnetic materials has a center point and is configured and dimensioned to vary the magnetic attraction as the opposite magnetic material is moved away from the center point.

35. The prosthesis of claim 34, wherein said at least one of said hard or soft magnetic material is configured and dimensioned in an annular shape.

36. The prosthesis of claim 34, wherein said at least one of said hard or soft magnetic material is configured and dimensioned with radiating petals.

37. The prosthesis of claim 34, wherein said at least one of said hard or soft magnetic material is configured and dimensioned with interlaced fingers.

38. The prosthesis of claim 34, wherein said at least one of said hard or soft magnetic material is configured and dimensioned with a varying thickness in a radial direction from the center point.

39. The prosthesis of claim 38, wherein said at least one of said hard or soft magnetic material is stepped.

40. The prosthesis of claim 34, wherein said at least one of said hard or soft magnetic material is configured and dimensioned to provide a plurality discrete spots of material arranged around the center point.

41. The prosthesis of claim 40, wherein said discrete spots of material vary in size along at least one radial direction with respect to the center point.

42. The prosthesis of claim 34, wherein one of said first and second components is operatively connected to the its corresponding bone portion across an articulating joint surface.

43. A prosthesis comprising:
a first component configured and dimensioned to carry an articulating surface of a joint;
a second component configured and dimensioned to interface with said first component on one surface and receive a bone of a joint on an adjacent surface;
first magnetic material disposed within said first component;
second magnetic material disposed within said second component wherein said second magnetic material is concentrated substantially centrally and around a periphery such that a magnetic attractive gradient is formed between said first component and said second component when said first component translates with respect to said second component; and
wherein said first magnetic material and said second magnetic material interact to maintain said first component in substantially an equilibrium position with respect to said second component.
